(19) United States
(12) Patent Application Publication
(10) Pub. No.: US 2009/0035233 A1
(43) Pub. Date: Feb. 5, 2009

(54) Conversion of liquid formulations to a powder

(75) Inventors: Ralph Spindler, Palatine, IL (US); Stephen J. Urbanec, Arlington Heights, IL (US); Rosario Oteri, Montvale, NJ (US)

Correspondence Address: MARSHALL, GERSTEIN & BORUN LLP, 233 S. WACKER DRIVE, SUITE 6300, SEARS TOWER, CHICAGO, IL, 60606, US

(73) Assignee: AMCOL International Corporation, Arlington Heights, IL (US)

(21) Appl. No.: 11/664,013

(22) PCT Filed: Oct. 4, 2005
(86) PCT No.: PCT/US05/35508

§ 371 Date: May 27, 2008

Related U.S. Application Data
(60) Provisional application No. 60/616,663, filed on Oct. 7, 2004.

(51) Int. Cl. A61K 8/81; A61K 47/32; A61Q 17/00; A61K 47/46

(52) U.S. Cl. 424/59; 514/772.4; 514/783

(57) ABSTRACT

A powdered composition comprising a liquid or semisolid formulation and a polymeric microparticle delivery system. The powdered composition is capable of converting to the liquid or semisolid composition upon application to a substrate, such as skin or a scalp.

### CROSS REFERENCE TO A RELATED APPLICATION

This application claims the benefit of U.S. provisional patent application Ser. No. 60/616,663, filed Oct. 7, 2004.

### FIELD OF THE INVENTION

The present invention is directed to conversion of a liquid or semisolid composition to a powdered form by entrapping the composition on a microparticle delivery system. The resulting powdered composition after application to the skin, or other substrate, provides the appearance and feel of applying the liquid or semisolid composition.

### BACKGROUND OF THE INVENTION

Many formulated personal care and home care products designed for consumer use are liquid or semisolid compositions, such as solutions, emulsions, and gels. These products are widely used to effectively and simply impart product characteristics that consumers desire. One disadvantage with such product forms is that they can be difficult to apply without spilling or wasting a portion of the product. In addition, the risk of accidental spillage is high. Therefore, it would be desirable to provide the same product, but in the form of a powder, such that after application to the skin, or other substrate, the powdered product imparts the feel, esthetics, and efficacy of the liquid or semisolid product, while overcoming disadvantages associated with a liquid or semisolid product.

### SUMMARY OF THE INVENTION

One aspect of the present invention is to adsorb a liquid or semisolid composition onto a microparticle delivery system to convert the liquid or semisolid product into a powder. A microparticle delivery system utilized in the present invention has a sufficient adsorption capacity to load a high concentration of the liquid or semisolid composition onto the delivery system, and therefore make the powdered composition more cost effective. A high load of a liquid or semisolid composition on a delivery system also ensures that the feel of the powdered composition after application is liquid-like to the user. In addition, the powdered composition preferably does not whiten or impart other negative esthetic effects to the skin or other substrate after application.

In another aspect of the present invention, liquid and semisolid compositions that can be adsorbed onto the microparticle delivery system include oil-in-water emulsions and water-in-oil emulsions, for example, lotions, and creams. The liquid or semisolid compositions also can be aqueous or nonaqueous formulations, for example, gels, wash products, and toners.

These and other aspects and novel features of the present invention will become apparent from the following detailed description of the preferred embodiments.

### DETAILED DESCRIPTION OF THE PREFERRED EMBODIMENTS

Delivery systems routinely are used in personal care and topical pharmaceutical compositions to extend the useful life of an active compound, to protect the active compound from decomposition in the composition, and/or to enable or facilitate formulation of the active compound into a composition due to problems such as solubility or esthetics. A delivery system that can provide all these advantages is the adsorbent microparticles.

One preferred class of adsorbent microparticle polymers useful as a delivery system is prepared by a suspension polymerization technique, as set forth in U.S. Pat. Nos. 5,677,407; 5,712,358; 5,777,054; 5,830,967; and 5,834,577, each incorporated herein by reference. Such an adsorbent polymer is sold under the tradename of POLY-PORE® E200, available from AMCOL International Corporation, Arlington Heights, Ill.

Another preferred class of adsorbent microparticle polymers useful as a delivery system is prepared by a precipitation polymerization technique, as set forth in U.S. Pat. Nos. 5,830,960 and 5,837,790, each incorporated herein by reference. Such an adsorbent polymer is sold under the tradename POLY-PORE® L200, also available from AMCOL International Corp.

These adsorbent microparticle polymers also can be modified after incorporation of an active compound to retard the rate of release of the active compound, as set forth in U.S. Pat. No. 6,491,953, incorporated herein by reference.

Still another preferred class of adsorbent microparticle polymer prepared by a precipitation polymerization technique and useful as a delivery system is disclosed in U.S. Pat. Nos. 4,962,170; 4,948,818; and 4,962,133, and sold under the tradename of POLYTRAP®, also available from AMCOL International Corp.

Other commercially available adsorbent polymers include, for example, MICROSPONGE® (a copolymer of methyl methacrylate and ethylene glycol dimethacrylate), available from Cardinal Health, Sommerset, N.J., and Poly-HIPE polymers (e.g., a copolymer of 2-ethylhexyl acrylate, styrene, and divinylbenzene) available from Biopore Corporation, Mountain View, Calif.

Preferred polymeric microparticle delivery systems comprise a copolymer of allyl methacrylate and ethylene glycol dimethacrylate, a copolymer of ethylene glycol dimethacrylate and lauryl methacrylate, a copolymer of methyl methacrylate and ethylene glycol dimethacrylate, a copolymer of 2-ethylhexyl acrylate, styrene, and divinylbenzene, and mixtures thereof.

In accordance with the present invention, the microparticle delivery system is a component of final powdered composition, which further contains an emulsion (either oil-in water or water-in-oil), a gel, or a solution formulation. The liquid or semisolid formulation incorporated onto the delivery system can be an aqueous or a nonaqueous composition. The microparticle delivery system typically is present in the powdered composition in an amount of less than about 25 wt. % of the final powdered composition.

As used herein, the term “formulation” means a fully formulated composition comprising a plurality ingredients selected from, but not limited to, water, organic solvents, topically active compounds, emulsifiers, pH adjusters, dyes, fragrances, buffers, skin care compounds, preservatives, chelating agents, viscosity modifiers, surfactants, fillers, pigments, dispersing agents, oils, and opacifying agents.

In further accordance with the present invention, the final powdered composition is a loaded microparticle delivery system that is a powder when applied to a substrate, either skin or other surface, and upon application to the substrate converts into a liquid-like product that has a feel substantially similar to the original liquid or semisolid composition that was loaded onto the delivery system.

Nonlimiting examples of substrates are the skin, the scalp, inanimate hard surfaces, and cloth. Typically, the powdered composition is converted to a liquid or semisolid composition by rubbing the powdered composition on a substrate during or after application, or by applying a similar force on a present powdered composition applied to a substrate.

A powdered composition of the present invention is prepared by adding a previously formulated liquid or semisolid composition to the microparticle delivery system. The addition is performed with sufficient mixing to provide a homogeneous powdered composition. In some cases, it may be necessary to dilute the liquid or semisolid composition with an appropriate amount of a solvent or carrier, for example, to reduce the viscosity of the liquid or semisolid composition. Typically, the solvent or carrier is identical to the solvent or carrier of the liquid or semisolid composition. The solvent or carrier can be hydrophilic or hydrophobic.

For example, a water-in-oil (w/o) emulsion can be diluted with a portion of the hydrophobic oil used to manufacture the emulsion, such as a silicone fluid that is used in many liquid foundation products. Also, an oil-in-water (o/w) emulsion can be diluted with water or other hydrophilic solvent (e.g., glycerin or propylene glycol). Such dilutions can be performed either by first adding the dilution solvent to the microparticle delivery system followed by addition of the liquid or semisolid composition, or by first diluting the liquid or semisolid composition with an appropriate amount of the solvent, then adding the mixture to the polymeric microparticles.

The hydrophobic oil used as a dilution solvent can be a natural oil including, but are not limited to, rice bran oil, lanolin oil, linseed oil, coconut oil, olive oil, menhaden oil, castor oil, soybean oil, tall oil, rapeseed oil, palm oil, neatsfoot oil, eucalyptus oil, peppermint oil, rose oil, clove oil, lemon oil, pine oil, orange oil, almond oil, apricot kernel oil, avocado oil, chaulmoogra oil, cherry pit oil, cocoa butter, cod liver oil, corn oil, cottonseed oil, egg oil, ethiodized oil, grape seed oil, hazel nut oil, hybrid safflower oil, hydrogenated castor oil, hydrogenated coconut oil, hydrogenated cottonseed oil, hydrogenated menhaden oil, hydrogenated palm kernel oil, hydrogenated palm oil, hydrogenated peanut oil, hydrogenated shark liver oil, hydrogenated soybean oil, hydrogenated vegetable oil, jojoba oil, mink oil, moring a oil, olive husk oil, palm kernel oil, palm oil, peach kernel oil, peanut oil, pengawar djambi oil, safflower oil, sesame oil, shark liver oil, shea butter, sunflower seed oil, sweet almond oil, vegetable oil, walnut oil, wheat bran lipids, wheat germ oil, and mixtures thereof.

The hydrophobic oil also can be a synthetic oil, like a hydrocarbon, e.g., mineral oil, 1-decene dimer, a polydecene, paraffin, petrolatum, or an isoparaffin, for example. Another useful class of synthetic oils is the silicone oils, like dimethicone, and the functional silicone oils, like dimethicone copolyol. The silicone oils have a viscosity of about 10 centipoise (cps) to about 600,000 cps, and typically about 350 cps to about 10,000 cps, at 25° C. Examples of silicone oils include dimethicone, dimethicone copolyol, dimethiconol, simethicone, phenyl trimethicone, stearoxy dimethicone, trimethylsilylamodimethicone, an alkyl dimethicone copolyol, and a dimethicone having polyoxyethylene and/or polyoxypropylene side chains.

The diluting solvent also can hydrophilic, like water or a water-soluble organic compound containing one to six, and typically one to three, hydroxyl groups, e.g., alcohols, diols, triols, and polyols. Specific examples of hydrophilic solvents include, but are not limited to, water, methanol, ethanol, isopropyl alcohol, n-butanol, n-propyl alcohol, ethylene glycol, propylene glycol, glycerol, diethylene glycol, dipropylene glycol, tripropylene glycol, hexylene glycol, butylene glycol, 1,2,6-hexanetriol, sorbitol, PEG-4,1,5-pentanediol, similar hydroxyl-containing compounds, and mixtures thereof.

The weight ratio of the liquid or semisolid composition to the microparticle delivery system is judiciously selected to provide a sufficient delivery of the composition and a sufficient esthetic feel after application of the powdered composition to the substrate. The amount of liquid or semisolid composition added to the microparticle delivery system, i.e., the “load” of the composition, also is related to the identity of the microparticle delivery system. Typically, load levels range from about a 1:1 load (i.e., weight ratio of the liquid or semisolid composition to the delivery system) to about an 8:1 load. The preferred load range is about 2:1 to about 7:1, and a more preferred load range is about 3:1 to about 7:1.

A powdered composition of the present invention can be a personal care, over the counter, pharmaceutical, color cosmetic treatment, or surface cleaning product, for example. The powdered composition can effectively delivery fragrances, pigments, skin treatment agents, topical drugs, and similar topically applied compounds, for example.

More particularly, a powdered composition of the present invention can be, but is not limited to, deodorants, skin-care compositions, antioxidants, insect repellants, counterirritants, steproids, retinoids, antibacterials, antifungals, antiinflammatories, antibiotics, topical anesthetics, sunscreens, skin rash, skin disease, and dermatitis medications, antiitch and irritationreducing compositions, antiseptics, antiacne preparations, diaper rash relief preparations, herpes treatments, pruritic medications, psoriasis, seborrhea, and scabicide compositions, skin-bleaching compositions, skin protectants, optical brighteners, and other cosmetic and medicinal topical compositions. A powdered composition also can be a cleaner or disinfectant for application to inanimate substrates, such as hard surfaces and cloth.

The present powdered compositions, therefore, can be, for example, baby lotions, baby creams, cosmetic basecoats and undercoats, blushers, cuticle softeners, depilatories, dusting and talcum powders, eye lotions, eye makeup products, eye makeup removers, eye shadows, eyebrow pencils, eyeliners, face powders, face, body, and hand creams and lotions, foot powders and sprays, cosmetic foundations, fragrance products, hormone creams and lotions, indoor tanning preparations, leg and body paints, lipsticks, makeup bases, makeup fixatives, makeup products, manicuring products, mascara, moisturizing creams and lotions, nail creams and lotions, nail extenders, nail polishes and enamels, night creams and lotions, antidandruff products, paste masks, perfumes, rouges, sachets, skin care products, skin fresheners, skin lighteners, suntan products, tonics, pharmaceutical skin treatment products, dressings, hair grooming aids, underarm deodorants, wave sets, and wrinkle smoothing creams and lotions.

### EXAMPLES

### Example 1

An o/w liquid foundation composition was loaded onto POLYTRAP® 6603 microparticles at load levels of 1 g/g (50% load), 3 g/g (75% load) and 4 g/g (80% load). At the lowest load level, the resulting powdered composition spreads smoothly on the skin, but turns chalky after drying. At the highest load level, the resulting powdered composition was more difficult to apply because it formed balls upon application to the skin. In a preferred embodiment, the POLYTRAP® 6603 first was loaded with 2 g/g of dimethicone (100 cst), then loaded with 2 g/g of the foundation composition. The resulting powdered composition had excellent skin feel, with minimal “balling” upon application to the skin. Alternatively, the liquid foundation composition first can be diluted with a silicone fluid, then the diluted foundation composition can be admixed with the microparticle delivery system to produce a final powdered composition having an excellent skin feel and minimal “balling.”

### Example 2

The following formulation was prepared:

| | | % by |
|---|---|---|
| Phase | Ingredient | Weight |
| A | Deionized Water (DI) | 41.80 |
| A | Tetrasodium EDTA | 0.05 |
| A | 2-Methyl-1,3-propanediol | 2.00 |
| A | Xanthan Gum (2% aqueous solution) | 10.00 |
| B | MAGNABRITE K (AMCOL) | 1.00 |
| C | Glycerin 96% | 4.00 |
| C | Deionized Water | 5.00 |
| C | Triethanolamine 99% | 0.75 |
| D | ARLACEL 165 (Uniquema) (Glyceryl Stearate and PEG- | 1.50 |
| | 100 Stearate) | |
| D | TRIVENT DOS (Trivent) (Dioctyl Sebacate) | 12.00 |
| D | TRIVENT PE-48 (Trivent) (Pentaerythritol Tetra-2- | 5.00 |
| | ethylhexanoate) | |
| D | ARLACEL 20 (Uniquema) (Sorbitan Laurate) | 2.50 |
| D | Cetearyl Alcohol | 1.50 |
| D | Stearic Acid | 2.50 |
| E | Mica MRP | 2.00 |
| E | Microna Matte White (Rona) | 4.00 |
| E | Microna Matte Yellow (Rona) | 2.00 |
| E | Microna Matte Orange (Rona) | 0.20 |
| E | Micronia Matte Red (Rona) | 0.20 |
| E | Micronia Matte Black (Rona) | 0.20 |
| F | Germaben II (International Specialty Products) | 1.00 |
| G | POLY-PORE ® E-200 (AMCOL) | 0.80 |
| | Total | 100.00 |

The composition was prepared by combining the ingredients of phase A at 75° C., then adding phase B and mixing until uniform. Then phase C was added to the resulting mixture. The ingredients of phase D were admixed, then heated to 75° C., and added to the mixture. Next, the ingredients of phase E were combined and added to the mixture. The resulting composition was cooled to 40° C., then phases F and G were added.

The resulting foundation composition (50 g) was added to 10 g of POLYTRAP® 6603 microparticles to provide a powdered composition that applied to the skin easily.

### Example 3

The following anhydrous gel was prepared and then entrapped in POLYTRAP® 6603 microparticles.

| Phase | Ingredient | % by weight |
|---|---|---|
| A | Castor oil | 76.48 |
| A | Capric/Caprylic Triglyceride | 20.00 |
| B | CABOSIL ® M-5 (Cabot) | 3.00 |
| B | Thixcin R (Elementis) (Trihydroxystearin) | 0.10 |
| B | BHT (Butylated Hydroxytoluene) | 0.02 |
| C | Retinol 50C (BASF) | 0.40 |
| | Total | 100.00 |

The ingredients of phase A were mixed and heated to 50° C. Then the ingredients of phase B were added to phase A, and the resulting composition was mixed at 50° C. until the powders dissolved. The mixture was cooled to 40° C., then phase C was added.

Fifteen grams of the resulting composition were added to POLYTRAP® 6603 microparticles, which provided a powder that dragged when applied to the skin, whereas adding 20 g of the composition to 5 g of POLYTRAP® 6603 provided a powder that applied to the skin smoothly.

### Example 4

The following o/w emulsion containing dihydroxyacetone was prepared.

| Phase | Ingredient | Weight % |
|---|---|---|
| A | DI water | 49.9 |
| A | Disodium EDTA | 0.1 |
| A | Xanthan Gum (2% aqueous solution | 15 |
| B | Cetearyl Alcohol | 3 |
| B | Caprylic/Capric Triglyceride | 12.4 |
| B | Octyldodecanol | 2 |
| B | Steareth-21 | 2.5 |
| B | Behenyl Alcohol | 2.5 |
| B | LIPOMULSE 165 (Lipo Chemical) (Glyceryl | 1.5 |
| | Stearate and PEG-100 Stearate) | |
| C | Phenonip (Clariant) | 1 |
| D | 50% Dihydroxyacetone in water | 10 |

The ingredients of phase A were admixed and heated to 75° C. The ingredients of phase B were admixed, then added to phase A and mixed with a homogenizer. The resulting mixture was cooled to 40° C., then phase C was added. The pH of the resulting mixture was adjusted to 5. After the mixture was cooled to room temperature, phase D was added. Fifty grams of the composition was added to 10 g of POLYTRAP® 6603 microparticles.

### Example 5

The following anhydrous foundation product was prepared.

| Material | Weight % |
|---|---|
| Titanium dioxide | 17.7 |
| Yellow iron oxide | 2.2 |
| Red iron oxide | 0.5 |
| Black iron oxide | 0.5 |
| Cyclomethicone | 48.3 |
| Diisopropyl adipate | 16.2 |
| MAGNASPERSE ® CM DIPA Gel (AMCOL) | 14.6 |
| Total | 100 |

The cyclomethicone and diisopropyl adipate were admixed, and the MAGNASPERSE® CM-DIPA (AMCOL Health and Beauty Solutions) was added using a dispersion blade. The resulting mixture was mixed until a homogeneous gel formed. The pigments were added in the order listed, and the final mixture was mixed using a homogenizer until the pigments were well dispersed and a uniform color formed in the gel.

The gel (30 g) was added to 5 g of POLYTRAP® 6603 microparticles and mixed until the product was homogeneous.

### Example 6

An acne cleanser was prepared as follows.

| Phase | Ingredient | Weight % |
|---|---|---|
| A | Water | 53.99 |
| A | Sodium C14-16 olefin sulfonate | 6.0 |
| A | CARBOPOL ® Ultrez 20 (Noveon) (Polymer | 0.75 |
| | acrylate/C10-30 Alkyl Acrylate Crosspolymer) | |
| B | Sodium cocoyl isethionate | 0.75 |
| C | Sodium hydroxide (50%) | 0.10 |
| D | POLY-PORE ® 145BP (AMCOL) - 15% aqueous | 37.41 |
| | dispersion containing benzoyl peroxide | |
| E | Phenonip (Clariant) | 1.0 |
| | Total | 100 |

The ingredients of phase A were admixed and heated to 65° C., then phase B was added to phase A. The resulting mixture was cooled to 45° C., then the POLY-PORE® 145BP dispersion was added with continued stirring. Phase E was added to the mixture, and the pH of the composition was adjusted with phase C to a final pH of 5.

The acne cleanser formulation (50 g) was added to 10 g of POLYTRAP® 6603 microparticles.

### Example 7

An acne repair formulation was prepared as follows.

| | | Weight |
|---|---|---|
| Phase | Ingredient | % |
| A | Water | 54.52 |
| A | CARBOPOL ® Ultrez 20 | 0.15 |
| A | Na₂EDTA | 0.1 |
| B | LIPOMULSE 165 (Lipo Chemical) | 1.5 |
| B | Cyclomethicone | 5 |
| B | Dimethicone (100 cst) | 1 |
| C | Phenonip (Clariant) | 1 |
| D | POLY-PORE ® 145BP (AMCOL) - 15% dispersion in | 36.73 |
| | water | |
| | Total | 100 |

The components of phase A were admixed, brought to pH 4.5 using 50% sodium hydroxide, then heated to 75° C. Phase B components were combined and heated to 75° C. Phase B was added to phase A, then the resulting mixture was admixed using a homogenizer. The resulting emulsion was cooled to 45° C., and while mixing and phase C and phase D were added. The pH of the emulsion was checked to ensure it was at pH 4.5

The resulting emulsion (50 g) was added to 10 g of POLYTRAP®6603 microparticles resulting in a powder-like product that felt similar to the emulsion when applied to skin.

Obviously, many modifications and variations of the invention as hereinbefore set forth can be made without departing form the spirit and scope thereof, and, therefore, only such limitations should be imposed as are indicated by the appended claims.

What is claimed is:

1. A powdered composition comprising a liquid or semisolid formulation and a polymeric microparticle delivery system, said powdered composition capable of converting to the liquid or the semisolid formulation upon application to a substrate.

2. The composition of claim 1 wherein the liquid or semisolid formulation is an emulsion, a gel, or a solution.

3. The composition of claim 1 wherein the liquid or semisolid formulation is an aqueous formulation or a nonaqueous formation.

4. The composition of claim 2 where the emulsion is an oil-in-water or a water-in-oil formulation.

5. The composition of claim 2 where the formulation comprises water, a hydrophilic liquid, or a mixture thereof.

6. The composition of claim 2 where the formulation comprises a hydrophobic liquid.

7. The composition of claim 1 wherein the polymeric microparticles are selected from the group consisting of a copolymer of allyl methacrylate and ethylene glycol dimethacrylate, a copolymer of ethylene glycol dimethacrylate and lauryl methacrylate, a copolymer of methyl methacrylate and ethylene glycol dimethacrylate, a copolymer of 2-ethylhexyl acrylate, styrene, and divinylbenzene, and mixtures thereof.

8. The composition of claim 1 wherein the polymeric microparticles comprise a copolymer of allyl methacrylate and ethylene glycol dimethacrylate, a copolymer of ethylene glycol dimethacrylate and lauryl methacrylate, or a mixture thereof.

9. The composition of claim 8 wherein the polymeric microparticles comprise copolymer of ethylene glycol dimethacrylate and lauryl methacrylate.

10. The composition of claim 1 wherein the weight ratio of the liquid or semisolid formulation to the polymeric microparticle delivery system is about 1:1 to about 8:1.

11. The composition of claim 10 wherein the weight ratio of the liquid or semisolid formulation to the polymeric microparticle delivery system is about 2:1 to about 7:1.

12. The composition of claim 11 wherein the weight ratio of the liquid or semisolid formulation to the polymeric microparticle delivery system is about 3:1 to about 7:1.

13. The composition of claim 1 wherein the substrate comprises skin, scalp, a hard surface, or cloth.

14. The composition of claim 13 wherein the substrate comprises skin or the scalp.

15. The composition of claim 1 further comprising a hydrophobic dilution solvent selected from the group consisting of rice bran oil, lanolin oil, linseed oil, coconut oil, olive oil, menhaden oil, castor oil, soybean oil, tall oil, rapeseed oil, palm oil, neatsfoot oil, eucalyptus oil, peppermint oil, rose oil, clove oil, lemon oil, pine oil, orange oil, almond oil, apricot kernel oil, avocado oil, chaulmoogra oil, cherry pit oil, cocoa butter, cod liver oil, corn oil, cottonseed oil, egg oil, ethiodized oil, grape seed oil, hazel nut oil, hybrid safflower oil, hydrogenated castor oil, hydrogenated coconut oil, hydrogenated cottonseed oil, hydrogenated menhaden oil, hydrogenated palm kernel oil, hydrogenated palm oil, hydrogenated peanut oil, hydrogenated shark liver oil, hydrogenated soybean oil, hydrogenated vegetable oil, jojoba oil, mink oil, moring a oil, olive husk oil, palm kernel oil, palm oil, peach kernel oil, peanut oil, pengawar djambi oil, rice bran oil, safflower oil, sesame oil, shark liver oil, shea butter, sunflower seed oil, sweet almond oil, vegetable oil, walnut oil, wheat bran lipids, wheat germ oil, a hydrocarbon, mineral oil, 1-decene dimer, a polydecene, paraffin, petrolatum, an isoparaffin, dimethicone, dimethicone copolyol, dimethiconol, simethicone, phenyl trimethicone, stearoxy dimethicone, trimethylsilylamodimethicone, an alkyl dimethicone copolyol, a dimethicone, and mixtures thereof.

16. The composition of claim 1 further comprising a hydrophilic dilution solvent selected from the group consisting of water, methanol, ethanol, isopropyl alcohol, n-butanol, n-propyl alcohol, ethylene glycol, propylene glycol, glycerol, diethylene glycol, dipropylene glycol, tripropylene glycol, hexylene glycol, butylene glycol, 1,2,6-hexanetriol, sorbitol, PEG-4,1,5-pentanediol, similar hydroxyl-containing compounds, and mixtures thereof.

17. The composition of claim 1 selected from the group consisting of a fragrance, a drug, a therapeutic agent, a deodorant, an antiperspirant, a skin conditioner, an antioxidant, an insect repellant, a counterirritant, a vitamin, a steroid, a skin-lightening composition, a self-tanning composition, an antibacterial composition, an antifungal composition, an antiinflammatory composition, a topical anesthetic, an epidermal lipid replacement, a sunscreen, an optical brightener, a dermatitis or skin disease medication, and mixtures thereof.

18. A method of treating mammalian skin comprising a step of contacting the skin with a powdered composition of claim 1.

19. The method of claim 18 wherein the powdered composition is converted to a liquid or a semisolid composition after application to the skin.

20. The method of claim 19 wherein the powdered composition is converted to a liquid or a semisolid composition by rubbing the composition on the skin.
